(19) United States
(12) Patent Application Publication
(10) Pub. No.: US 2016/0367290 A1
(43) Pub. Date: Dec. 22, 2016

(54) NASAL MUCOUS REMOVAL APPARATUS

(71) Applicant: Charla Baker-Jackson, Los Angeles, CA (US)

(72) Inventor: Charla Baker-Jackson, Los Angeles, CA (US)

(21) Appl. No.: 15/161,584

(22) Filed: May 23, 2016

Related U.S. Application Data
(60) Provisional application No. 62/165,047, filed on May 21, 2015.

(51) Int. Cl. A61B 17/50; A61B 17/221

(52) U.S. Cl. CPC A61B 17/50; A61B 17/221; A61B 2017/2212

(57) ABSTRACT

A nasal mucous removal apparatus including: a proximal loop and at least one distal loop, wherein at least one of the proximal loop and the at least one distal loop comprise one or more pieces of bendable material and are connected.

### CROSS-REFERENCE TO RELATED PATENT APPLICATION

This application claims priority from provisional patent application No. 62/165,047, filed on May 21, 2015, in the United States Patent and Trademark Office, the disclosure of which is incorporated herein in its entirety by reference.

### BACKGROUND

1. Field

Exemplary embodiments relate to an apparatus for the removal of dried nasal mucous (boogers). More specifically, exemplary embodiments relate to a wire (or bendable, non-sharp material) device, designed to be inserted into an orifice to remove/pull out hardened mucous/debris that is difficult to access or remove safely via finger, fingernail, suction device, or any other removal device designed for an orifice.

2. Description of the Related Art

Any discussion of the related art throughout the specification should in no way be considered as an admission that such related art is widely known or forms part of common general knowledge in the field.

The removal of mucous from an infant and/or small child's nose is necessary in order to keep the nasal airway open and clear of any debris, thus improving the child's ability to breathe comfortably.

Removing hardened nasal mucous and debris without the abrasive entry, vacuum-like suction, and without scraping or significantly irritating the orifice tissue are two of several advantages which render the instant inventive concept different from other products. A number of products have been made and/or attempted to be made that remove debris from the nasal cavity, but fail to do so in a non-invasive, non-aggressive manner. Additionally, existing products are not effective in the removal of hardened mucous that may be stationary and attached to the wall of an orifice, like a nostril. For instance, U.S. Pat. No. 4.403,611 to Babbitt et al. (1983) is a suction device described as “a portable, self-cleaning or flushing evacuator apparatus” that aspirates and removes fluids from a nasal cavity and/or anatomical orifice via forceful suction. This reference, however, may not effectively remove hardened mucous.

Product size and/or bulkiness is also a significant factor when considering the fragility and miniscule size of an infant's or small child's orifice and/or nasal cavity. U.S. Pat. No. 4,422,456 to Tiep (1983), U.S. Pat. No. 4,681,571 to Nehring (1987), U.S. Pat. No. 5,183,467 to Mouney (1993), U.S. Pat. No. 5,246,666 to Vogler et al. (1993), U.S. Pat. No. 5,257,633 Vogler et al. (1993), U.S. Pat. No. 5,320,812 to Harper (1994), U.S. Pat. No. 5,326,535 to Vogler et al. (1994), U.S. Pat. No. 5,344,611 to Vogler et al. (1994), U.S. Pat. No. 5,533,506 to Wood (1996), U.S. Pat. No. 6,238,377 to Liu, U.S. Pat. No. 6,736,792 to Liu (2004), U.S. Pat. No. to Teel (1941), U.S. Pat. No. 2,890,699 to Miller (1959), U.S. Pat. No. 4,487,336 to Sneider (1984), U.S. Pat. No. 5,114,415 to Shedlock (1992), U.S. Pat. No. 6,290,667 to Cook (2001), and 2007/0270736 to Giarrocco-Brettner (2007) are all larger in size relative to an inventive concept disclosed in this document, and many include two separate ends that disconnect for cleaning purposes; whereas an inventive concept disclosed in this application is small, compact in size, and can easily be reused when rinsed and cleaned with a disinfectant solution (e.g. rubbing alcohol, antibacterial soap or sanitizer), and transported in multi-sized objects (e.g. diaper bag, pocket, a toiletry case, etc.).

Exemplary embodiments of the present inventive concept may overcome the above-mentioned disadvantages and other disadvantages not described above. Also, an exemplary embodiment is not required to overcome the disadvantages described above, and an exemplary embodiment may not overcome any of the problems described above.

### SUMMARY

An aspect of the exemplary embodiment is to provide users with a product which comprises a single strip (or multiple strips connected together) of bendable wire (or flexible, adaptable, smooth, non-sharp non-abrasive material) with two (or more) loops—a small loop on the proximal end that is inserted into a nostril to secure and remove hardened nasal mucous from the nostril; and, a second (or more), ring-like sized loop(s) on the distal end, designed for a finger or multiple fingers to hold the apparatus and control the small loop's depth of insertion into the orifice/nostril/nasal cavity.

The distal end loop(s) can be covered by a material (e.g. rubber, or any flexible or moldable material) that assists with finger grip, thus granting a user more control while lessening the likelihood that the proximal loop end will go too deep into an orifice, extending beyond the intended area within a nostril, nasal cavity or orifice.

The above object, purposes, features, and advantages of the present invention are readily apparent from the enclosed detailed description of the best mode(s) for carrying out the invention when taken in connection with the accompanying drawings/figures which offer illustrative details of the embodiments.

### BRIEF DESCRIPTION OF THE DRAWINGS

Exemplary embodiments of the inventive concept will be more clearly understood from the following detailed description taken in conjunction with the accompanying drawings in which:

FIG. 1 is a perspective view of the nasal mucous removal apparatus having a single or adjoining/interconnected construction between a proximal and distal loop, according to an exemplary embodiment;

FIG. 2 is a perspective view of the nasal mucous removal apparatus, according to an exemplary embodiment, in usage by an adult with a finger or fingers holding the apparatus during the removal of hardened nasal mucous from the nostril of an infant and/or small child.

FIG. 3 is a perspective view of the nasal mucous removal apparatus, according to an exemplary embodiment, with hardened nasal mucous on the tip of the proximal loop.

### DETAILED DESCRIPTION OF EXEMPLARY EMBODIMENTS

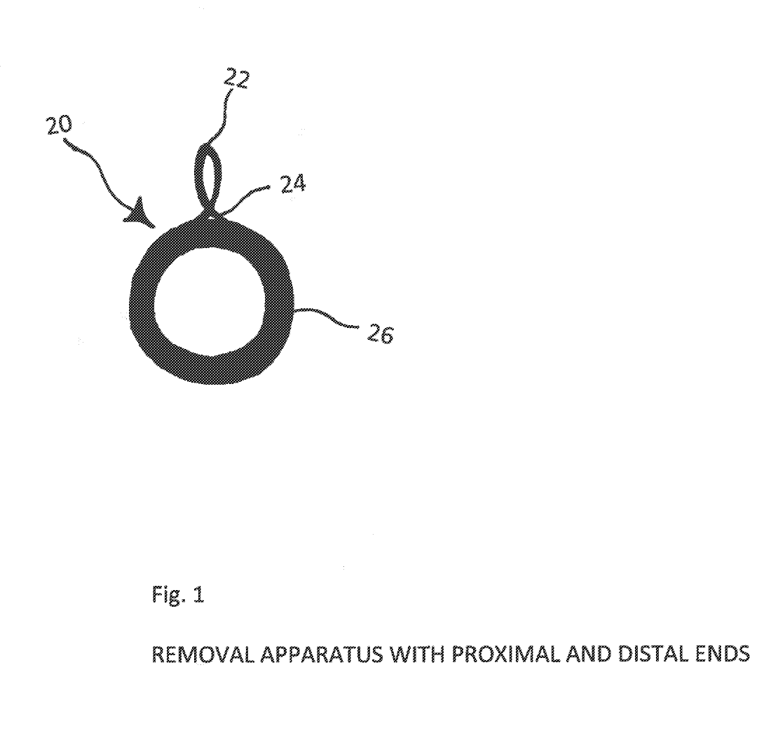

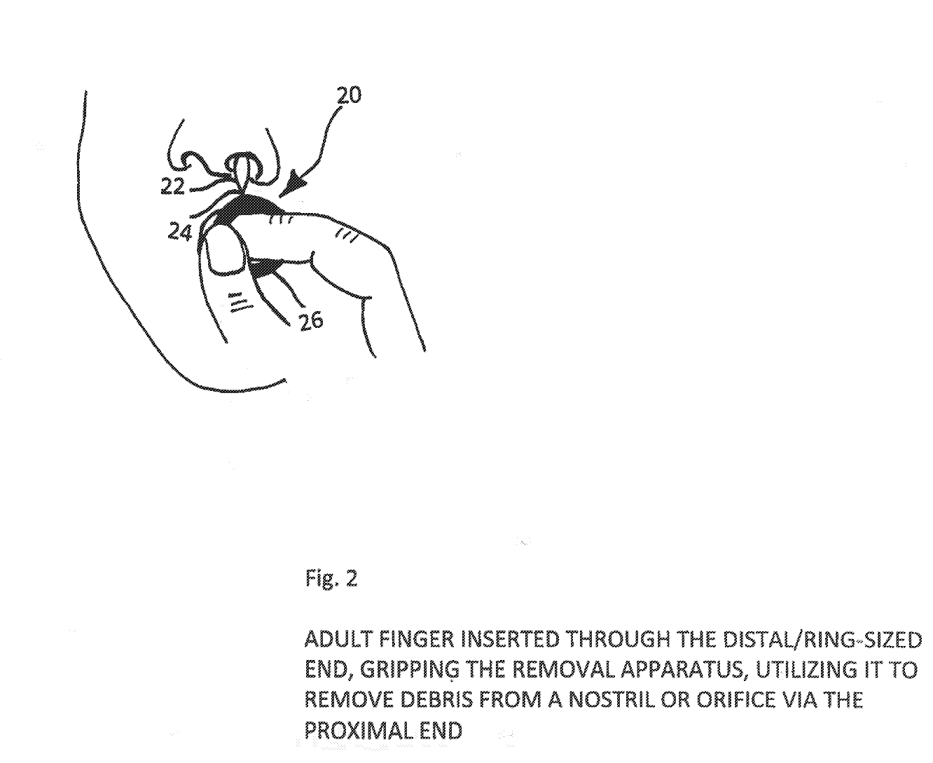

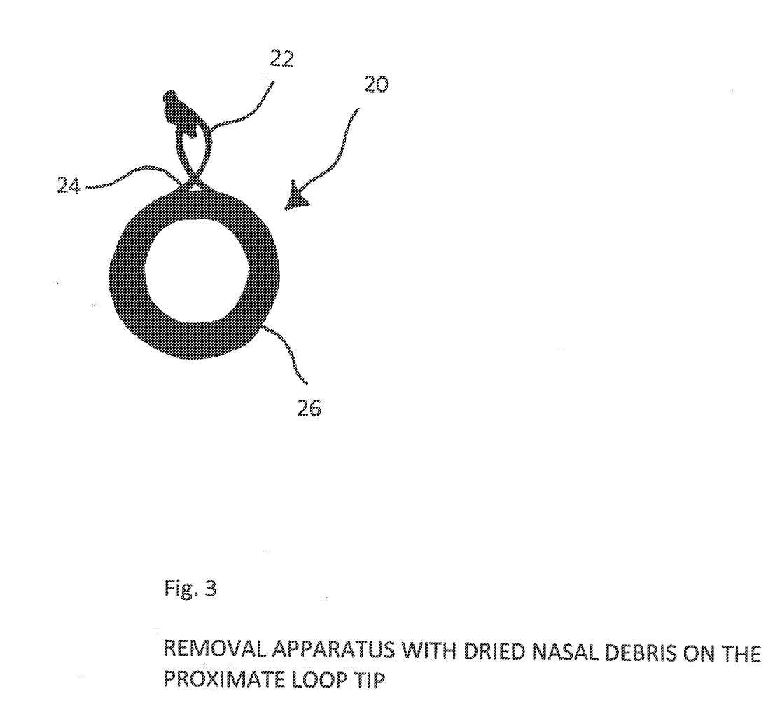

Referring to FIGS. 1, 2, and 3, a nasal mucous removal apparatus 20 is shown as a single object, in use by an adult user to remove dried nasal mucous from an orifice, and shown as a single object with the dried nasal mucous on the tip of the proximal loop. Nasal mucous removal apparatus 20 may be made out of smooth artistic wire (or a bendable, non-sharp material) with loops (at least one on the proximal 22 and distal 24 ends of the apparatus) and its measurements may vary slightly. The nasal mucous removal apparatus 20 material can be cleaned with a disinfectant (rubbing alcohol) and can be reused. The nasal mucous removal apparatus can include a material covering 26, preferably soft rubber or cotton, but can be any other material that can grip the wire (or a bendable, non-sharp material) on the distal loop 24 end of the apparatus.

The nasal mucous removal apparatus 20 can be fully utilized on its own as an individual, independent device (as shown in FIGS. 1, 2, and 3). For ultimate security, a finger or fingers (preferably index and/or middle finger) can wrap around the wire (or a bendable, non-sharp, non-abrasive material) via the material covering 26 on the distal loop 24 end of the apparatus to lessen the likelihood that the proximal loop 22 will be inserted too deeply into an orifice, thus granting a user more control and ensuring that the device does not extend beyond the intended area within a nostril, nasal cavity or orifice.

What is claimed is:

1. A nasal mucous removal apparatus comprising:
a proximal loop; and
at least one distal loop,
wherein at least one of the proximal loop and the at least one distal loop comprise one or more pieces of bendable material, and
wherein the proximal loop and the at least one distal loop are connected.

2. The nasal mucous removal apparatus of claim 1, wherein the proximal loop is insertable into a human orifice to remove mucous.

3. The nasal mucous removal apparatus of claim 1, wherein at least one of the proximal loop and the at least one distal loop comprise a bendable, non-sharp, non-abrasive material.

4. The nasal mucous removal apparatus of claim 1, wherein at least one of the proximal loop and the at least one distal loop comprise a wire.

5. The nasal mucous removal apparatus of claim 1, wherein the proximal loop and the at least one distal loop are connected substantially in the middle of the nasal mucous removal apparatus.

6. The nasal mucous removal apparatus of claim 1, wherein the at least one distal loop comprises a covering that can be gripped by a finger or fingers.
